(19) United States
(12) Patent Application Publication
(10) Pub. No.: US 2020/0019726 A1
(43) Pub. Date: Jan. 16, 2020

(54) Systems and Methods for Secure Medical Communication

(71) Applicants: Randy Perecman, Richboro, PA (US); Richard M. Evans, Suffern, NY (US)

(72) Inventors: Randy Perecman, Richboro, PA (US); Richard M. Evans, Suffern, NY (US)

(21) Appl. No.: 16/035,796

(22) Filed: Jul. 16, 2018

(51) Int. Cl. G06F 21/62; G16H 10/60; H04W 4/14; G16H 80/00; G06F 21/40; G06F 21/44

(52) U.S. Cl. CPC G06F 21/6245; G16H 10/60; H04W 4/14; G16H 70/20; G06F 21/40; G06F 21/44; G16H 80/00

(57) ABSTRACT

Embodiments relate to systems and methods for secure medical communication. The system can be implemented in a hospital or other health care setting. Recipient devices, such as smartphones, associated with family members or other authorized users can be registered to a notification engine to receive automatic distributions of patient updates or other medical information entered for the patient by doctors, nurses, or other care providers. In implementations, the entire system is secure and capable of compliance with privacy or other regulations, such as HIPPA or others. In implementations, the entry of an update to the patient's medical information can trigger the delivery of a text message or other notification of that update to the set of registered devices, on an immediate or automatic basis The recipients are accordingly relieved of any need to call in to a hospital unit or take other positive actions to receive medical information.

### CROSS-REFERENCE TO RELATED APPLICATION

This application claims the benefit, including priority, of U.S. Provisional Application 62/533,226 filed Jul. 17, 2017, entitled “Systems and Methods for Secure Medical Communication,” by the same inventors herein, which application is incorporated by reference in its entirety.

### FIELD

The present teachings relate to systems and methods for secure medical communication, and more particularly, to platforms and techniques for communicating updates on a hospital or other patient's medical status to family members or other registered users, on a timely, secure, and convenient basis, including on a one-way or forward-transmission basis.

### BACKGROUND

In the medical field, access to timely information regarding a patient's condition can have an effect on the perception of quality of care. Family members, friends, colleagues, and other acquaintances of a patient frequently want to know the status of a patient's up-to-date condition, test results, medications, recovery progress, and/or other updates at various times of night or day.

However, in traditional hospital and other health care settings, the dissemination of medical updates is often carried out using cumbersome, untimely, and/or antiquated methods. Family members may be directed, for instance, to contact a doctor or nurse providing care for a patient through a telephone call to the floor during strictly defined operating hours, often ending in the middle evening hour. Manual inquiries by phone call may possibly be provided with basic privacy protection such as an assigned patient code, which may or may not be easy or convenient for family members to remember or record. When a patient's condition can change hour by hour or even more quickly, such mechanisms are not always sufficient to convey the most up-to-date medical state of the patient, including during late night, early morning, or other times. Health care providers also may not be available when a family member or other interested party attempts to call.

It may be desirable to provide systems and methods for medical communication, in which patient updates and related information can be received from authorized care providers and registered for secure, automatic dissemination via mobile devices or other channels, to desired recipients, such as by text messages to mobile phones or other channels to those family members or others. In implementations, the privacy of the medical information can be protected by avoiding the permanent storage of that information on cellular phones or other recipient devices.

### DESCRIPTION OF DRAWINGS

The accompanying drawings, which are incorporated in and constitute a part of this specification, illustrate embodiments of the present teachings and together with the description, serve to explain the principles of the present teachings. In the figures:

FIG. 1 illustrates an overall communications platform, in which systems and methods for secure medical communication can be implemented, according to various embodiments;

FIG. 2 illustrates a flow chart of registration processing, according to various embodiments;

FIG. 3 illustrates a flow chart of communications operations, according to various embodiments;

FIG. 4 illustrates a flow diagram of communications operations, in various further regards;

FIG. 5 illustrates exemplary hardware, software, and other resources that can be employed in a notification engine or other components, according to various implementations; and

FIG. 6 illustrates various control logic that can be hosted in a notification engine, according to various aspects.

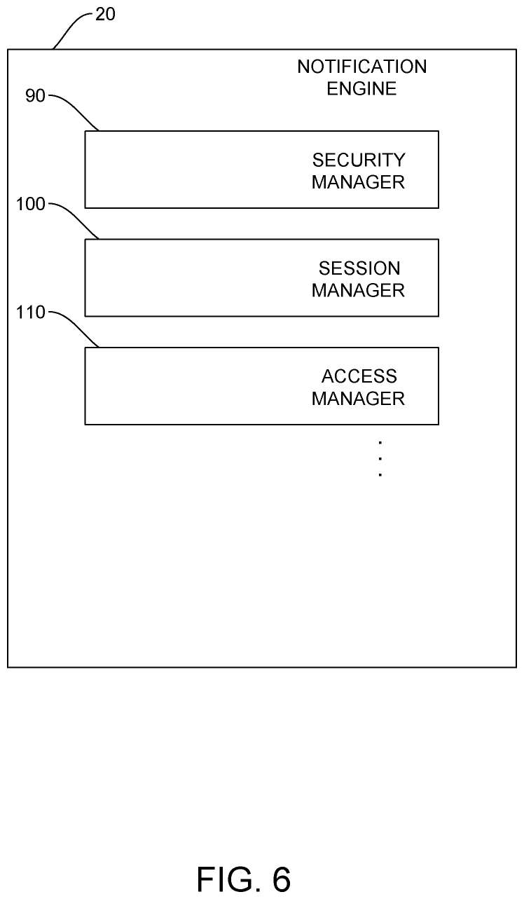

### DESCRIPTION

Embodiments of the present teachings relate to systems and methods for secure medical communication. More particularly, embodiments relate to platforms and techniques for promptly and securely distributing patient updates and/or other medical information, to family members or other authorized recipients using messaging to mobile smartphones or other mobile or networked devices from the patient's authorized care provider or providers. In aspects, systems and methods according to the present teachings allow family members or others to become registered to receive medical information for a given patient, and doctors, nurses, or other care providers to register to the platform and supply medical information regarding the subject patient.

The medical information can include, for instance, information such as updates to any of a variety of information including patient status, milestones, surgery or related procedures, rehab, locations in a hospital room or other facility, test results, transfers within or outside a facility, medications, admission or discharge, and/or other information or updates. In implementations, a doctor or other care provider can be encouraged or required to maintain a short or compact format for the medical information, and reserve more detailed reports for face to face or telephonic interactions.

The medical information can for example be communicated, transmitted or broadcast to the set of registered recipients using text messages and/or other channels, e.g., text messages delivered via a smartphone application, browser, via Short Messaging Service, FaceBook™, Twitter™, or other messages, or using other formats services, or channels.

Since the medical information can be automatically distributed to the set of registered recipients, the need for family members or others to frequently call, email, or otherwise contact the hospital or other facility, reach the correct unit or care provider, present codes or credentials, and finally verbally receive that type of information is eliminated, and patient and family awareness and experience can be improved. In addition, the updates or other medical information can be preserved as a text or other record, creating an accurate history of the patient progress, history, test results, medications, and other details of their treatment or overall medical encounter. It may be noted that in implementations, the system and methods of the invention can be configured to maintain medical privacy in accordance with applicable legal/regulatory requirements, such as HIPPA, California CMIA (Cal. Civ. Code §§ 56-56.37) or other state, federal, or other requirements.

Reference will now be made in detail to exemplary embodiments of the present teachings, which are illustrated in the accompanying drawings. Where possible the same reference numbers will be used throughout the drawings to refer to the same or like parts.

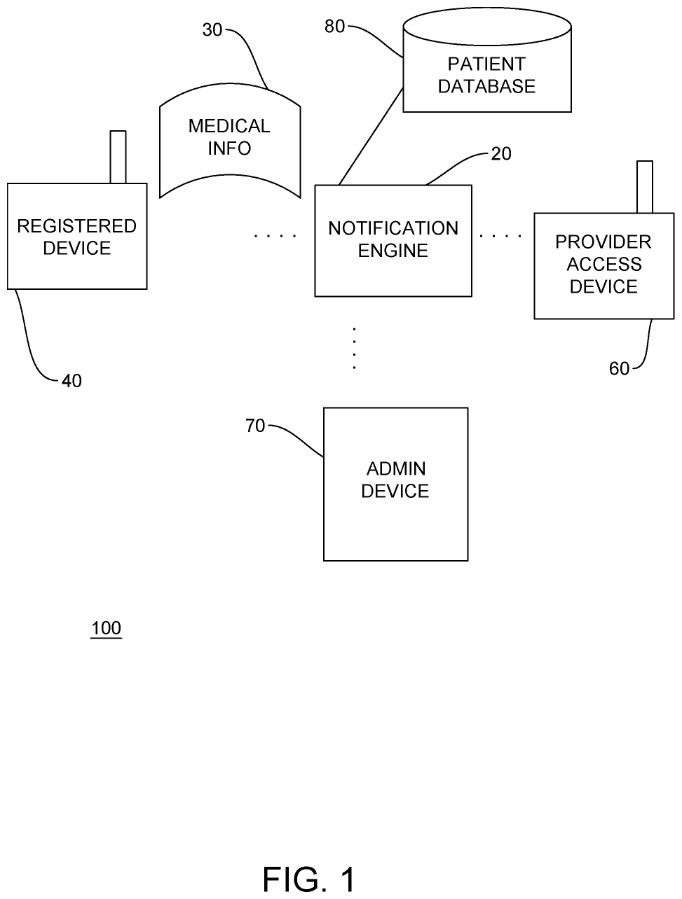

FIG. 1 illustrates an overall platform in which systems and methods for secure medical communication can operate, according to aspects. In aspects as shown, the overall platform can include a notification engine 20, such as a server or network-accessible service to transmit and manage messages including medical information 30 to a registered device 40 (or set of such devices) operated by a corresponding registered recipient 50. In aspects, the notification engine 20 can be implemented and/or operate in a network, such as a cloud-based network, and/or other network or service, such as merely for example, the FireCloud™ service operated by Google, Mountain View Calif., a service or affiliate of Alphabet Inc., Mountain View Calif. Other cloud-based or other networks, services, or facilities can be used. In aspects, the notification engine 20 can be or include hardware infrastructure such as a server or server farm or other collection or combination of servers, computers, or other processing resources, and/or can be or include network-accessible services. In aspects, merely for further example, the notification engine 20 and/or other processing resources or logic can be hosted by an off-site or remote third party, and/or can he hosted and/or operated by the associated hospital or other facility or organization. Other arrangements are possible.

In aspects, the registered device 40 can be or include one or more network-enabled wireless devices such as smartphones, tablets, and/or laptop or other computers, using for example the Google Android™ or Apple iOS™ operating platforms. The overall systems and methods can likewise use other devices such as smart watches, fitness devices, GPS units, pagers, and/or other devices configured to access and communicate with the notification engine 20.

Generally speaking, the overall platforms and methods of the present teachings designed to broadcast or distribute the medical information 30 to the registered recipients on a prompt, secure, timely and continuous or near-continuous, real-time or near-real-time, and/or other timely basis, so that those individuals can remain apprised of the medical condition of the patient in an up-to-date manner without having to take separate positive actions to access those updates or information each time. In implementations, the distribution and communication of medical information 30 can be managed by a session manager 100, which as illustrated in FIG. 7 in implementations can be hosted or implemented in notification engine 20, and/or in other local or remote hardware, services, or resources, such as participating mobile devise. In implementations, the session manager 100 can detect a message stream from a doctor, nurse, and/or other care provider, and automatically delete sessions from the care provider's patient list if that message stream has been dormant or inactive for a predetermined time period, such as 24, 36, 48, or other predetermined numbers of hours.

When configured to delete such sessions, session manager 100 and/or other logic can store data from medical information on a temporary or permanent basis, while removing the subject patient from the care prover's active or current patient list. In implementations, the message containing medical information 30 can itself be set to be deleted or disappear, for example, upon confirmation of receipt by the intended recipient, the expiration of predetermined time periods, and/or based on other conditions. It may similarly be noted that in implementations, the session manager 100 and overall system can be configured to permanently store and/or maintain medical information 30 and/or other data in a persistent state, for example, by archiving or storing that information in cloud-based storage and/or other storage resources. Among other things, this persistent data maintenance can allow the medical information 30 to be accessed or examined by a doctor or others at a later time, for instance to consider a patient's long-term medical history, to refresh clinical data, and/or perform other tasks.

Further, in implementations, the session manger 100 can be configured to detect stagnant or inactive message sessions, and for example to notify a doctor, nurses, or other care provider with a display on their patient list showing stagnant status, hours or minutes since the last update to medical information 30, and/or other information, to for instance prompt the care provider to enter fresh or updated medical information 30, without necessarily deleting the associated message session.

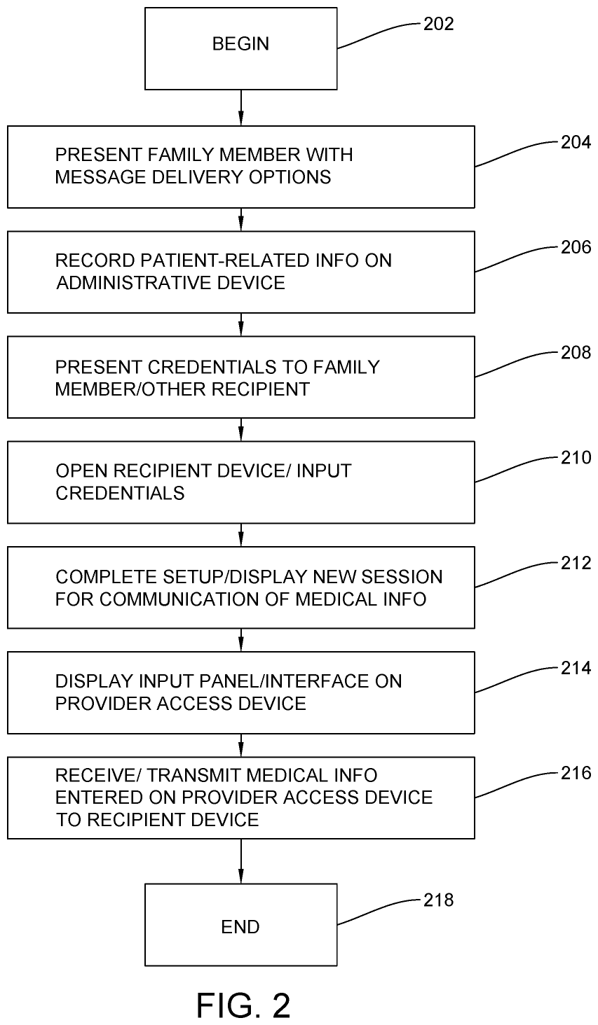

That information can be managed on a fully secure basis, and only distributed to authorized recipients to one or more registered device, such as smartphones used by authorized recipient(s) that have successfully completed a registration process such as that illustrated in FIG. 2 or otherwise. In implementations, and as for example shown in FIG. 7, security policies can be implemented, enforced, and or applied by a security manager 90, which can as illustrated be hosted or implemented in notification engine 20 and/or hosted or implemented in other local or remote hardware, services, or resource

It will be appreciated that In aspects, security can be achieved, applied or enforced using other or further techniques for account, device, and/or user authentication or verification, such as, in general, two-factor authentication techniques or others. Data such as a user's date of birth, for example, can be used. Other security mechanisms or techniques can be used, and in aspects, multiple security mechanisms can be used or enforced together.

In implementations, an authorized recipient can register or link more than one registered device 40, if desired. In implementations, the recipient can be presented with options to store, save, forward, and/or otherwise transmit a given messaging session for archival purposes, for example by storing the session to a cloud-based service, hard drive, and/or other storage, service, and/or location. In implementations, the patient, an administrator, and/or other participant can be provided with options to deactivate a selected recipient, at times or under conditions of their choosing, such as, simply for example, discharge of the subject patient. An authorized recipient can likewise, in embodiments, be provided with options to manually deactivate their own messaging service for a given patient at any chosen time.

As illustrated in FIG. 2, authorized recipients can be registered during a visit to the patient's hospital or other facility, or at other times. In 202, processing can begin. In 204, a family member, friend, or other desired recipient can be presented with a message delivery option regarding a patient of interest, for instance by a nurse, admissions personnel, and/or other person or persons associated with the hospital or other care facility. At that time, the prospective recipient can for instance be advised that the medical information to be delivered will be confidential and protected for purposes of HIPPA or other regulations. In implementations, the subject patient may be queried to approve or authorize delivery of medical information to the recipient.

In 206, the nurse or other hospital personnel can record patient-related information on, or input that information via, an administrative device 70, such as a smartphone, tablet, laptop, and/or other network-enabled device. In 208, patient-related information can be entered or recorded on the administrative device 70. The patient-related information can include, for example, patient name, date of birth, age, social security number, prescription data, and/or other identifying medical, clinical, and/or other information.

In 208, credentials for registration can be presented to the family member or other desired recipient. For example, an administrator, and/or other personnel can turn the administrative device 70 toward to the recipient to present a QR code (bar code) and/or other credentials or information. It may be noted that authorizing administrator or other non-medical personnel to register authorized recipients can relieve doctors or other care providers from that type of IT and other potentially time-consuming overhead. In 210, the family member or other individual can open the registered (or recipient) device 40 and input the registration credentials, such as for example by scanning the QR code on the registered device 40.

In 212, upon verification of the credentials or other information, the setup or registration of the registered device 40 can be completed. In implementations, a new message session displaying medical information 30 for the patient of interest can be immediately or later launched, for instance by displaying a text message on registered device 40, opening a browser or other application screen on registered device 40, and/or taking other actions to activate, present, or display medical information 30 and/or other data or information to the authorized recipient using the registered device 40. It may be noted that in implementations, the notification engine 20 and/or associated logic can be configured to present or display the medical information 30 on the recipient device 40, without permanently storing or displaying that information on the registered device 40, in aspects, to enhance the security, compliance, and/or privacy of medical information 30.

In 214, an input panel or interface can be displayed to a doctor, nurse, and/or other care provider on provider access device 60, such as a smartphone, tablet or laptop or other computer, to prompt the care provider to input or enter medical information 30, such as additions or updates to the patient's condition, status, surgical events, location, test results, prescribed medications, and/or other data related to the subject patient. In implementations, the doctor, nurse, and/or other care provider or participant can be presented or provided with a message template, for example, a set of blank or prefilled text messages and/or other information or formats containing headers, fields, or other structures of formats to express or capture medical information 30.

In implementations, the authorization or registration of either or both of authorized care providers and/or the registered recipients can be organized and managed by an access manager 110, which in implementations as shown in FIG. 7 can be hosted or implemented in the notification engine 20, and/or other local or remote hardware, resources, or services, including participating mobile devices.

For instance, it may be noted that in cases, more than one care provider can be authorized to enter medical information 30 for a subject patient, for example, an original or attending physician, followed by a second doctor such as a specialist to whom the subject patient may be transferred. The second doctor or other care provider or providers can for instance be added by the first doctor, by an administrator, and/or other authorized or registered user on the hospital or other care provider side. Thus in cases, multiple senders can join a one-way or other message session to intended recipients. In implementations, likewise, more than one provider access device 60 can be registered and/or used to enter medical information 30.

In 216, the medical information 30 entered on provider access device 60 can be received in notification engine 20 and/or other logic, and transmitted to the registered device 40. The medical information 30 can for example be managed by access manager 110 and transmitted to and displayed on the registered device 40 using, for example, an application installed on the registered device 40, such as a smartphone application or other application or software. In aspects, the medical information 30 can be transmitted to designated recipients who are not in the local geographic area of the patient or care facility, so that, for example, medical information 30 for a patient located in New York can be transmitted to one or more registered device 40 in California, or other locations. The medical information 30 can, in cases, likewise or instead be transmitted and displayed by text message, a display in a Web browser or other application, a tweet, and/or other message, channel, or format.

In implementations, it may be noted that the access manager 110 can manage not only the outbound medical information 30, but can also manage the access to the system by care providers and others, on the message intake or other side, so that, for example, a doctor, nurse, administrator, and/or other care provider or participant can log into the system using interfaces or portals on Web-based desktop browsers, mobile applications including browsers, and/or other local or remote and/or mobile or non-mobile hardware, resources, and/or services.

In aspects, a care provider can be presented with a similar work flow in both mobile and non-mobile formats, merely for instance, using a tablet or smartphone, or a desktop computer, in which after login the doctor or other care provider can be presented with a drop-down or other list of active patients, and links or other selection choices to send medical information 30 and/or related messages, including pre-formatted templates, to the patient's designated recipients. In implementations, the notification engine 20 and/or associated logic can be configured to transmit the entered medical information 30 immediately to the registered device 40, in “push” or broadcast fashion.

In implementations, the notification engine 20 and/or associated logic can in addition or instead be configured to transmit the medical information 30 to the patient, him or herself. In those cases, the patient can have or use their own registered device 40, which can again be or include various mobile or non-mobile devices, such as a mobile phone, tablet, laptop computer, desktop computers, and/or others. In a hospital or other patient care setting or facility, a patient can be assigned a tablet device which is placed in a hospital room and then registered to the patient, allowing a doctor, nurse, administrator, and/or other care provider or participant to communicate medical information 30 an/or other information to the patient, directly. In cases, the assigned tablet and/or other device can be updated if the patient is moved to a new room, or can continue to be sent to the same registered device 40 to “follow” the patient while located in the facility, if desired. Other arrangements are possible.

In implementations, the medical information 30 can be displayed to the desired recipient in read-only and/or one-way fashion, or in implementations can be presented as a two-way conversation or other message stream to which the authorized recipient can respond if desired. It may be appreciated that when chosen, a one-way configuration can offer advantages to the hospital or other organization hosting the notification engine 20 and/or other resources or services, including ease of installation, use, possibly reduced hardware requirements, and ease of maintenance and support, since a one-way platform can generally represent a more streamlined implementation compared to a multi-point, two-way, and/or other more elaborate platform. Other configurations and communication options can be used. In 218, processing can return to a prior processing point, jump to further processing point, or end.

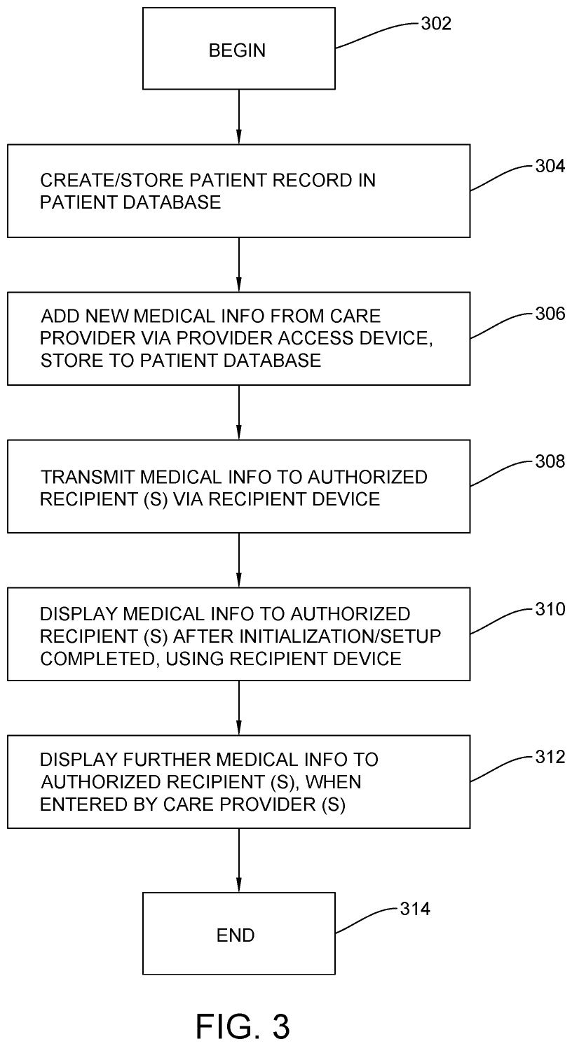

FIG. 3 illustrates a flowchart of messaging other processing that can be performed in systems and methods for secure medical communication, according to aspects. In 302, processing can begin. In 304, a new or revised patient record can be created and/or stored in patient database 80, for instance, by manual entry by a care provider. In 306, new or revised medical information 30 can be added by a care provider, administrator, and/or other participant via provider access device 60 and/or other input device, channel, or service.

In 308, medical information 30 can be transmitted to the registered device 40, and/or other device, channel, or service. In implementations, as noted, the nature of the communication can be one-directional so that medical information 30 is sent to one or more authorized recipient or recipients, in broadcast fashion. It will be appreciated, however, that in other implementations, the notification engine 20, recipient device 40 and/or other resources can be configured to allow two-way or other types of communication, so that, for instance, the recipient can type in a question to a care provider upon receipt of the medical information 30, and/or send other messages at other times. It will likewise be appreciated that while in implementations, messaging can take place solely between a care provider and the intended recipient(s). In further implementations, the smartphone application or other logic on recipient device 40 can be configured to allow communication or messaging between other parties, such as between two or more authorized recipients (such as family members), directly, to allow those parties for instance to discuss the medical information 30. Other communication arrangements are possible.

It may likewise be noted that while message described as containing or consisting of text messages, in implementations, the medical information 30 and/or other content can be or include non-textual information, such as pictures, video, sound, test result data, and/or other information or media. Still further, in embodiments, the set of authorized recipients can be broken into groups or categories, so that, for instance, certain groups can be designated to receive certain types or categories of medical information 30. For example, a spouse may be designated to receive medical information 30 related to necessary treatment decisions, and/or other types of medical information 30.

In 310, the medical information 30 can be displayed to the authorized recipient(s) after initialization or setup of their account or access is otherwise verified or completed, the recipient(s) using the registered device 40, and/or other device, channel, or service. In 312, further, additional, and/or updated data in medical information 30 can be displayed to the authorized recipient(s) on a real-time or near real-time basis, for instance, triggered by entry of that information by a care provider or other personnel.

In 314, processing can repeat, return to a prior processing point, jump to a further processing point, or end.

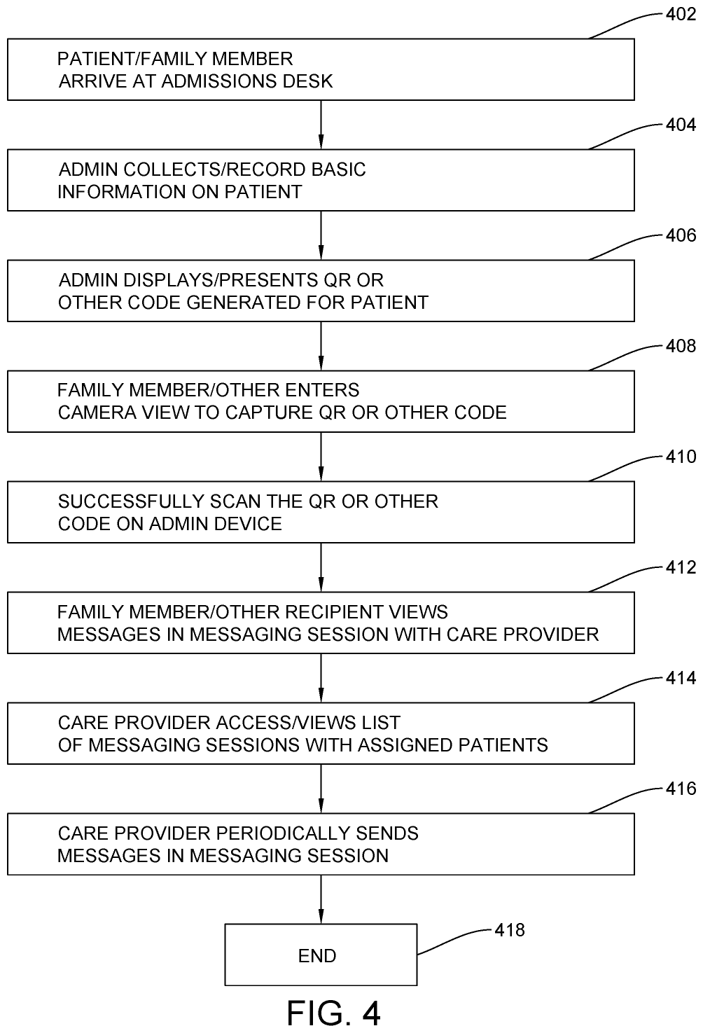

FIG. 4 illustrates platform configuration znc communications processing 400, including registration and messaging operations, according to various implementations of the present teachings, in further regards. In 402, processing can begin when a patient, family member or members, and/or other party arrives at an admissions desk, station, administrative office, and/or other location for intake. In 404, an administrator and/or other type pf personnel can collect and record basic information on a newly admitted patient, such as name, date of birth, social security information, attending doctor information, and/or other information, That information can for example be inquired from the patient or others verbally, and for instance captured in a standardized database or spreadsheet.

In 406, the administrator and/or other type of personnel can present of display a QR or other graphical code that may be generated for that patient, for example, using a tablet or other device turned to the patient, family member, or others. In 408, the family member or other participant can initiate a camera view to scan or capture the QR code, or other graphical or security code, which can verify that the patient or other user has authenticated the transmission of medical information using the subject platform. In 410, the QR or other code can be authenticated on the administrator's device, such as by detecting that code on a tablet or other device. In 412, an initial or further message from a doctor or other care provider can be displayed and/or viewed by the authenticated family member, for example by the display of textual messages on a smart phone or other device.

In 414, a doctor or other care provider can access and/or view a list or other summary of existing or pending conversations or messaging sessions for their patients of responsibility, during a duty shift or at other times. In 416, the doctor or other care provider can send a new message or update in the messaging session to the patient, family member, or other participant. In 418, processing can repeat, return to a prior processing point, jump to a further processing point, or end.

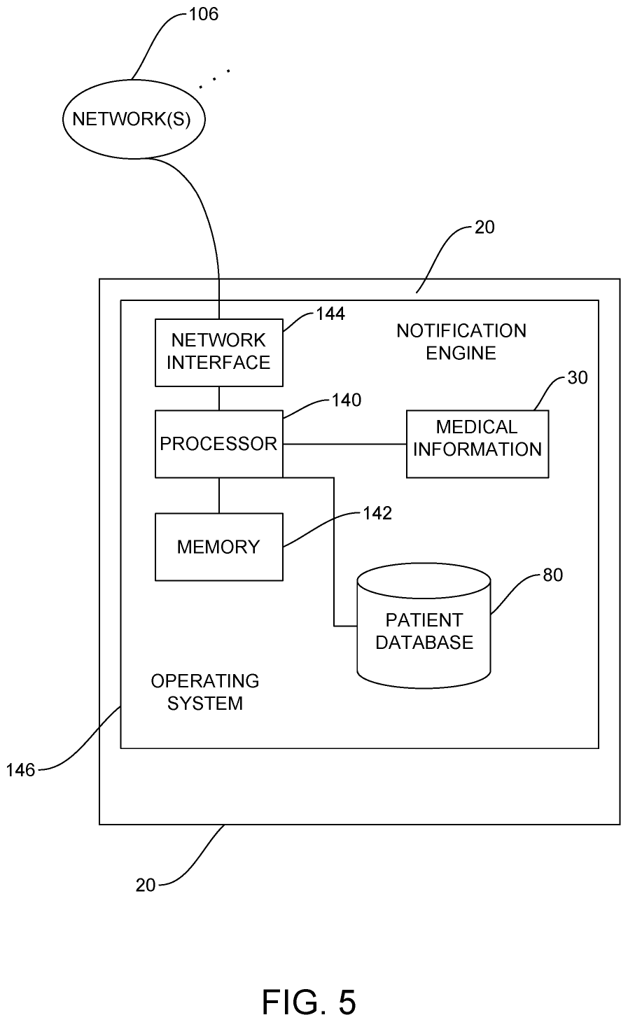

FIG. 5 illustrates various hardware, software, and other resources that can be used in a notification engine in implementations of medical communication, according to embodiments. In embodiments as shown, the notification engine 20 can comprise a server, farm, cluster, cloud-based network, or other platform including processor 140 communicating with memory 142, such as electronic random access memory, operating under control of or in conjunction with an operating system 146. The processor 140 in embodiments can be incorporated in one or more servers, and/or other computers, logic, or hardware resources, and/or as noted can be implemented using cloud-based resources. The operating system 146 can be, for example, a distribution of the Linux™ operating system, the Unix™ operating system, or other open-source or proprietary operating system or platform. The processor 140 can communicate with the patient database 80, such as a database or data store stored on a local hard drive or drive array, to access or store medical information 30, and/or subsets of selections thereof, along with other content, media, or other data. The processor 140 can further communicate with a network interface 144, such as an Ethernet or wireless data connection, which in turn communicates with the one or more networks 106, such as the Internet or other public or private networks to reach one or more registered device 40. The processor 140 can, in general, be programmed or configured to execute control logic and to control various processing operations, including to generate messaging operations according to the present teachings as described herein. In aspects, other controls or logic herein can be or include resources similar to those of the notification engine 20, and/or can include additional or different hardware, software, and/or other resources. Other configurations of the notification engine 20, associated network connections, and other hardware, software, and service resources are possible.

The foregoing description is illustrative, and variations in configuration and implementation may occur to persons skilled in the art. For example, while instances have been described in which a registered recipient is set up to receive medical information 30 for one patient, in cases, a registered recipient can be configured to receive medical information regarding two or more patients. For further example, while embodiments have been described in which the messaging platform is hosted or supported by one notification engine 20, in implementations, two or more notification engines 20 can be used. Other resources described as singular or integrated can in embodiments be plural or distributed, and resources described as multiple or distributed can in embodiments be combined. The scope of the present teachings is accordingly intended to be limited only by the following claims.

What is claimed is:

1. A method of delivering medical information, comprising:
registering a set of recipient devices in a notification engine;
receiving medical information from an authorized care provider for a patient; and
transmitting the medical information to the set of recipient devices, using one-way communication without permanently storing the medical information on the set of recipient devices.

2. The method of claim 1, wherein the set of recipient devices comprises at least one of—
a smartphone,
a tablet device,
a laptop computer, or
a desktop computer.

3. The method of claim 2, wherein the medical information is transmitted to the set of recipient devices via a text message.

4. The method of claim 2, wherein the medical information transmitted to the set of recipient devices comprises at least one of—
image information,
audio information, and
video information.

5. The method of claim 1, wherein the registering comprises using two-factor authentication to authenticate at least one user of the set of recipient devices.

6. The method of claim 1, wherein the authorized care provider comprises at least one of—
a doctor,
a nurse,
a pharmacist, or
an administrator.

7. The method of claim 1, wherein the medical information comprises information related to at least one of—
patient status,
patient milestones,
surgery status,
rehab protocols,
patient location,
test results,
patient medication, or
patient admission or discharge,

8. The method of claim 1, wherein the transmitting is triggered by the receipt of the medical information from the authorized care provider

9. The method of claim 1, further comprising storing the medical information.

10. The method of claim 1, generating a notification of stagnant status to the authorized care provider based on predetermined criteria applied to a messaging session with the set of recipient
